(19) United States
(12) Patent Application Publication
(10) Pub. No.: US 2008/0089928 A1
(43) Pub. Date: Apr. 17, 2008

(54) LIPOSOME DRUG CARRIERS WITH PH-SENSITIVITY

(76) Inventor: Stavroula Sofou, New York, NY (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 11/762,635

(22) Filed: Jun. 13, 2007

Related U.S. Application Data
(60) Provisional application No. 60/804,630, filed on Jun. 13, 2006.

(51) Int. Cl. A61K 9/127; A61K 47/00; A61P 43/00

(52) U.S. Cl. 424/450; 514/789

(57) ABSTRACT

A liposome composition for delivering of a biologically active agent having at a least first rigid lipid and a second rigid lipid each having a head group and a hydrophobic tail; a polyethyleneglycol-linked lipid having a side chain matching at least a portion of the first or the second lipid; and an entrapped biologically active agent. The first lipid can be a zwitterionic lipid and the second lipid can have a titratable head group. The composition can be adapted to release the entrapped at a certain pH.

### STATEMENT OF RELATED APPLICATIONS

This application is based on and claims priority on U.S. Provisional Patent Application No. 60/804,630 having a filing date of 13 Jun. 2006.

### BACKGROUND OF THE INVENTION

1. Technical Field

This invention generally relates to the field of drug carriers and more particularly relates to the field of using liposomes to deliver biologically active agents to cells, including cancer or tumor cells.

2. Prior Art

Liposomes, which are spherical, self-enclosed vesicles composed of amphipathic lipids, have been widely studied and are employed as vehicles for in vivo administration of therapeutic agents. Liposomes are structures defined by a phospholipid bilayer membrane that encloses an aqueous compartment. The membrane acts as a barrier that inhibits free molecular diffusion across the bilayer. The physicochemical characteristics of the liposome can be helpful in achieving certain effects from the liposome. Manipulation of these characteristics can have marked effects on the in vivo behavior of liposomes and can have a major impact on therapeutic success.

Liposomes and their potential as drug-delivery vehicles have been investigated for many years. Hydrophilic drugs, such as aminoglycosides, can be encapsulated in the internal aqueous compartments, whereas hydrophobic drugs may bind to or are incorporated in the lipid bilayer. The bilayers are usually composed of natural or synthetic phospholipids and cholesterol, but the incorporation of other lipids or their derivatives, as well as proteins, is also possible.

Accordingly, there is always a need for an improved drug carrier and improved liposome compositions. It is to these needs, among others, that this invention is directed.

### BRIEF SUMMARY OF THE INVENTION

Briefly, this invention includes a liposome composition for delivering a bioactive agent, comprising at a least a first lipid and a second lipid each having a head group; a polyethyleneglycol-linked lipid having a tails matching at least a portion of the first lipid or the second lipid; and an entrapped biologically active agent. The composition is adapted to release the entrapped biologically active agent at a certain pH.

Another embodiment includes a method of formulating a therapeutic liposome composition having sensitivity to a target cell. The method includes selecting a liposome formulation composed of pre-formed liposomes having an entrapped biologically active agent; selecting from a plurality of targeting conjugates a targeting conjugate composed of a lipid having a polar head group and a hydrophobic tail, a hydrophilic polymer having a proximal end and a distal end, and a targeting ligand attached to the distal end of the polymer; and combining the liposome formulation and the selected targeting conjugate to form a therapeutic, target-cell pH sensitive liposome composition.

These features, and other features of this invention, will become more apparent to those of ordinary skill in the relevant art when the following detailed description of the preferred embodiments is read in conjunction with the appended drawings in which like reference numerals represent like components throughout the several views.

### BRIEF DESCRIPTION OF THE DRAWINGS

FIG. 1A shows that liposomes composed of equimolar DPPC and DSPA lipids exhibited a pH-dependent content release.

FIG. 1B shows that liposomes composed of 75% mole DPPC and 25% mole DSPA lipids exhibited a pH-dependent content release.

FIG. 1C shows that liposomes composed of 90% mole DPPC and 10% mole DSPA lipids exhibited a pH-dependent content release.

FIG. 2A shows the pH-dependent release of fluorescent contents (calcein) from liposomes (equimolar DPPC DSPA lipid ratio) in 10% serum supplemented media.

FIG. 2B shows the pH-dependent release of fluorescent contents (calcein) from liposomes (75% mole DPPC and 25% mole DSPA) in 10% serum supplemented media.

FIG. 2C shows the pH-dependent release of fluorescent contents (calcein) from liposomes (90% DPPC and 10% DSPA) in 10% serum supplemented media.

FIG. 2D shows that release of fluorescent contents (calcein) from liposomes (25% DSPA lipid and 75% DSPC lipid) in the presence of 10% serum proteins.

FIG. 3A shows content release from liposomes over 5 days.

FIG. 3B shows content release from liposomes upon pH decrease after a 60 minute preincubation period at pH 7.4.

FIG. 4A shows the thermal scans of PEGylated liposomes composed of equimolar DPPC and DSPA lipids (with 5% mole cholesterol).

FIG. 4B shows that longer incubation of the liposome suspensions resulted in shifts toward higher thermal transitions with decreasing pH.

FIG. 5 shows the thermographs of equimolar DPPC- and DSPA-containing liposomes at neutral pH in the presence and absence of lysozyme.

### DEFINITIONS

Unless specifically indicated otherwise, all technical and scientific terms used herein have the same meaning as commonly understood by those of ordinary skill in the art to which this invention belongs. For purposes of the present invention, the following terms are defined.

A “pH-sensitive” lipid refers to a lipid whose ability to change the net charge on its head group depends at least in part on the pH of the surrounding environment.

“Biologically active agents” as the term is used herein refers to molecules which affect a biological system. These include molecules such as proteins, nucleic acids, therapeutic agents, vitamins and their derivatives, viral fractions, lipopolysaccharides, bacterial fractions, and hormones. Other agents of particular interest are chemotherapeutic agents, which are used in the treatment and management of cancer patients. Such molecules are generally characterized as antiproliferative agents, cytotoxic agents, and immunosuppressive agents and include molecules such as taxol, doxorubicin, daunorubicin, vinca-alkaloids, actinomycin, and etoposide.

“Liposome” as the term is used herein refers to a closed structure comprising an outer lipid bi- or multi-layer membrane surrounding an internal aqueous space. In particular, the liposomes of the present invention form vase-like structures which invaginate their contents between lipid bilayers. Liposomes can be used to package any biologically active agent for delivery to cells.

### DETAILED DESCRIPTION OF THE PREFERRED EMBODIMENTS

Embodiments of this invention include pH-sensitive liposomes of a specific composition forming a stable structure that can efficiently carry biologically active agents. More particularly, the liposome can contain one or more biologically active agents, which can be administered into a mammalian host to effectively deliver its contents to a target cell or tumor cell. The liposomes are capable of carrying biologically active agents, such that the agents are sequestered in one environment and can be selectively exposed in another.

Liposome Composition

One embodiment of this invention is a pH-sensitive liposome composition for delivering a biologically active agent, comprising:

a) at least a first lipid and a second lipid each having a head group and a hydrophobic tail;

b) a polyethyleneglycol-linked lipid having a tail matching at least a portion of the first or the second lipid; and

c) an entrapped biologically active agent.

The composition is adapted to release the entrapped biologically active agent at a certain pH. In one example, one of the lipids is a zwitterionic lipid and one of the lipids is a titratable head group forming lipid. It is understood that additional lipids also can be incorporated into the composition.

Liposomes suitable for use in the composition include those composed primarily of vesicle-forming lipids. Such a vesicle-forming lipid is one that (a) can form spontaneously into bilayer vesicles in water, as exemplified by the phospholipids, or (b) is stably incorporated into lipid bilayers, with its hydrophobic moiety in contact with the interior, hydrophobic region of the bilayer membrane, and its head group moiety oriented toward the exterior, polar surface of the membrane. Many lipids suitable with this embodiment are of the type having two hydrocarbon chains, typically acyl chains, and a head group, either polar or nonpolar. There are a variety of synthetic lipids and naturally forming lipids, including the phospholipids, such as DPPC, DSPA, DPPA, and DSPC, where the two hydrocarbon chains are typically at least 16 carbon atoms in length. The vesicle-forming lipids of this type are preferably ones having two hydrocarbon chains, typically acyl chains, and a head group, either polar or nonpolar.

The pH-sensitive liposome can be selected to achieve a specified degree of fluidity or rigidity, to control the stability of the liposome in serum, to control the conditions effective for insertion of the targeting conjugate, as will be described, and to control the rate of release of the entrapped biologically active agent in the liposome. Liposomes having a more rigid lipid bilayer, or a gel phase bilayer, are achieved by incorporation of a relatively rigid lipid, e.g., a lipid having a relatively high phase transition temperature, e.g., above about 41° C. Rigid, i.e., saturated, lipids contribute to greater membrane rigidity in the lipid bilayer. Other lipid components, such as cholesterol, are also known to contribute to membrane rigidity in lipid bilayer structures. In contrast, lipid fluidity can be achieved by incorporation of a relatively fluid lipid, typically one having a lipid phase with a relatively low liquid to gel crystalline phase transition temperature, e.g., at or below working temperature (e.g. body temperature).

These liposomes can contain titratable domain-forming lipids that phase-separate in the plane of the membrane as a response to decreasing pH values resulting in pH-controlled release of encapsulated contents. In one embodiment, the liposomes are comprised of two lipid types (both T_{g}>37° C.): one type is a zwitterionic rigid lipid (e.g., dipalmitoyl phosphatidyl choline, DPPC, T_{g}=41° C.), and the other component is a ‘titratable domain-forming’ rigid lipid (e.g. distearoyl phosphatidic acid, DSPA, T_{g}=75° C.) that is triggered to phase-separate in the plane of the membrane as a response to decreasing pH values. At physiological pH (7.4) the lipid-headgroups of the ‘domain-forming’ rigid lipid are charged, electrostatic repulsion should prevail among DSPA lipids, and the liposomal membrane would appear more mixed and homogeneous, resulting in stable retention of encapsulated contents.

The lipid phase-separation can be tuned by introducing a titratable charge on the headgroups of the domain-forming lipids. The extent of ionization on the headgroups of the domain-forming lipids can be controlled by using the pH to adjust the balance between the electrostatic repulsion among the headgroups and the van der Waals attraction among the hydrocarbon chains. The longer-hydrocarbon chain lipids that could phase-separate and form domains can be selected to have titratable acidic moieties on the head group (e.g., phosphatidic acid). At neutral pH, the headgroups of these lipids are negatively charged opposing close approximation and formation of domains. As the pH is decreased, gradual head group protonation minimizes the electrostatic repulsion and lipid domains are formed.

In one embodiment, one of the lipids of the liposome can have a negatively charged head group. A negatively charged head group can help reduce the likelihood of the liposome sticking to other cells when in the blood stream. In this embodiment, the liposomes comprise ionizable ‘domain-forming’ (‘raft’-forming) rigid lipids that are triggered to form domains as a response to the endosomal acidic pH. Domain formation (or else lateral lipid-separation) at the endosomal pH can cause the encapsulated contents to be released probably due to imperfections in ‘lipid packing’ around the domain ‘rim’. At physiological pH (during circulation) the lipids are charged, the liposome membrane may be ‘mixed’ so that the contents cannot leak. At the acidic endosomal pH (5.5-5.0), domain-forming lipids become increasingly protonated (non-ionized) and lipid domains of clustered protonated lipids can form resulting in release of encapsulated contents. In one embodiment, the lipids can have a pK value between about 3 and about 7.0. In another embodiment, the lipids can have a pK value between about 5 and about 5.5.

In another embodiment, the liposomes disclosed herein may further comprise stabilizing agents or have an aqueous phase with a high pH. Examples of stabilizing agents are a phosphate buffer, an insoluble metal binding polymer, resin beads, metal-binding molecules or halogen binding molecules incorporated into the aqueous phase to further facilitate retention of hydrophilic therapeutic modalities. Additionally, liposomes may comprise molecules to facilitate endocytosis by the target cells.

Biologically Active Agents

In one embodiment, the liposomal encapsulation of a biologically active agent enhances the bioavailability of the modalities in cancer cells. In this embodiment, the liposome can be used to encapsulate a biologically active agent (e.g., cancer modalities) and efficiently release the therapeutic modality in cancer cells, thus allowing toxicity to occur in the tumor cells. For example, the use of pH sensitive liposome allows more complete release of the therapeutic modalities upon endocytosis by the cancer cell.

The liposome can have a phospholipid-membrane rigidity to improve the retention of the bioactive agent in the liposome during blood circulation. The addition of PEG also reduces liposome clearance, thus increasing liposome accumulation in tumors. For example, one embodiment includes a pH-sensitive liposome with rigid membranes that combine long circulation times with the release of contents in the endosome. Other types of pH-sensitive liposomes can include charged titratable peptides on the surface that can cause phase separation and domain formation on charged membranes.

Biologically active agents suitable with such liposomes include but are not limited to natural and synthetic compounds having the following therapeutic activities: anti-arthritic, anti-arrhythmic, anti-bacterial, anticholinergic, anticoagulant, antidiuretic, antidote, antiepileptic, antifungal, anti-inflammatory, antimetabolic, antimigraine, antineoplastic, antiparasitic, antipyretic, antiseizure, antisera, antispasmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic, and vasodilating. In one illustrative example, the entrapped agent is a cytotoxic drug, that is, a drug having a deleterious or toxic effect on cells.

Administration of Liposome Composition

Another embodiment of this invention includes a method comprising pre-injecting the individual with empty liposomes and saturating the reticuloendothelial organs to reduce non-tumor specific spleen and liver uptake of the liposome-encapsulated therapeutics upon administration thereof.

In use and application, the liposome can be used to preferentially deliver a biologically active agent to a target cell or cancer cell. For example, in drug delivery to metastatic tumors with developed vasculature, the preferential tumor accumulation and retention of liposomes is primarily dependent on their size (EPR effect), and can result in adequate tumor adsorbed doses.

The liposome of the invention may be formulated for parenteral administration by bolus injection or continuous infusion. Formulation for injection may be presented in unit dosage form in ampoules, or in multi-dose containers with an added preservative. The compositions may take such forms as suspensions, solutions or emulsions in oily or aqueous vehicles, and may contain formulatory agents such as suspending, stabilizing, and/or dispersing agents. Alternatively, the active ingredient may be in powder form for reconstitution with a suitable vehicle, e.g., sterile pyrogen-free water, before use.

One embodiment includes a method for administering a biologically active agent comprising selecting a liposome comprising at a least first rigid lipid and a second rigid lipid each having a head group and a hydrophobic tail and a polyethyleneglycol-linked lipid having a side chain matching at least a portion of the first or the second lipid, wherein the first lipid is a zwitterionic lipid and the second lipid has a titratable head group; and the composition is adapted to release the entrapped at a certain pKa; preparing a liposome composition with the at least the first rigid lipid and the second rigid lipid and the polyethyleneglycol-linked lipid; preparing a therapeutic liposome by combining the composition with a biologically active agent so that the biologically active agent is within the liposome composition whereby the therapeutic liposome is adapted to release the entrapped at a certain pKa; and administering the therapeutic liposome to a subject.

The liposomes according to the invention may be formulated for administration in any convenient way. The invention therefore includes within its scope pharmaceutical compositions comprising at least one liposomal compound formulated for use in human or veterinary medicine. Such compositions may be presented for use with physiologically acceptable carriers or excipients, optionally with supplementary medicinal agents. Conventional carriers can also be used with the present invention.

Overcoming Immune Response

To overcome immunogenicity, the liposomes can be modified for use with the specific organism by those with ordinary skill in the art. In another embodiment, a method further comprises coating the outer membrane surfaces of the liposomes with molecules that preferentially associate with a specific target cell. These molecules or targeting agents may be antibodies, peptides, engineered molecules, or fragments thereof.

For example, to achieve tumor targeting of ovarian and breast cancer cells and internalization, liposomes can be coated (immunolabeled) with Herceptin, a commercially available antibody that targets antigens that are over-expressed on the surface of such cancer cells. Herceptin is chosen to demonstrate proof of principle with the anticipation that other antibodies, targeting ovarian, breast, liver, colon, prostate and other carcinoma cells could also be used. The target cells may be cancer cells or any other undesirable cell. Examples of such cancer cells are those found in ovarian cancer, breast cancer or metastatic cells thereof. The active targeting of lipsomes to specific organs or tissues can be achieved by incorporation of lipids with monoclonal antibodies or antibody fragments that are specific for tumor associated antigens, lectins, or peptides attached thereto.

Because the biologically active agent is sequestered in the liposomes, targeted delivery is achieved by the addition of peptides and other ligands without compromising the ability of these liposomes to bind and deliver large amounts of the agent. The ligands are added to the liposomes in a simple and novel method. First, the lipids are mixed with the biologically active agent of interest. Then ligands are added directly to the liposomes to decorate their exterior surface.

Preparing Liposomes

The liposomes may be prepared by a variety of techniques, such as those detailed in Lasic, D. D. Liposomes from Physics to Applications. Elsevier, Amsterdam (1993), and specific examples of liposomes prepared in support of the present invention will be described herein. Typically, the liposomes can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates have sizes between about 0.1 to 10 microns.

After formation, the liposomes are sized. One more effective sizing method for liposomes involves extruding an aqueous suspension of the liposomes through a series of polycarbonate membranes having a selected uniform pore size in the range of 0.03 to 0.2 micron, typically about 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less. In one embodiment of the present invention, the liposomes are extruded through a series of polycarbonate filters with pore sizes ranging from 0.2 to 0.08 μm resulting in liposomes having diameters in the approximate range of about 120 nm.

Incorporating Biologically Active Agent into Liposomes

The biologically active agent of choice can be incorporated into liposomes by standard methods, including passive entrapment of a water-soluble compound by hydrating a lipid film with an aqueous solution of the agent, passive entrapment of a lipophilic compound by hydrating a lipid film containing the agent, and loading an ionizable drug against an inside/outside liposome pH gradient. Other methods, such as reverse evaporation phase liposome preparation, are also suitable.

Another embodiment includes a method of formulating a therapeutic liposome composition having sensitivity to a target cell. The method includes selecting a liposome formulation composed of pre-formed liposomes having an entrapped biologically active agent; selecting from a plurality of targeting conjugates a targeting conjugate composed of a lipid having a polar head group and a hydrophobic tail, a hydrophilic polymer having a proximal end and a distal end, and a targeting ligand attached to the distal end of the polymer; and combining the liposome formulation and the selected targeting conjugate to form a therapeutic, target-cell pH sensitive liposome composition.

The following examples serve to illustrate further the present invention.

### EXAMPLES

In the following examples, the constituent lipids were selected to both have long saturated hydrocarbon-chains of different lengths, the shorter being mostly in the gel phase (T_{g}=41° C.) and the longer being in the gel phase (T_{g}=75° C.) at the working temperature (37° C.), so that domain formation would have persistent lipid packing ‘defects’ along the domain/non-domain interface and cause increased membrane permeability and release of encapsulated contents. The choice of lipids having high T_{g} values was thought to be more useful in drug delivery applications.

The lipids for use in these examples include 1,2-Dipalmitoyl-sn-Glycero-3-Phosphocholine (DPPC), 1,2-Dipalmitoyl-sn-Glycero-3-Phosphate (Monosodium Salt) (DPPA), 1,2-Distearoyl-sn-Glycero-3-Phosphocholine (DSPC), 1,2-Distearoyl-sn-Glycero-3-Phosphate (Monosodium Salt) (DSPA), 1,2-Dilauroyl-sn-Glycero-3-Phosphocholine (DLPC), 1,2-Dipalmitoyl-sn-Glycero-3-Phosphoethanolamine-N-[Methoxy(Polyethylene glycol)-2000] (Ammonium Salt) (DPPE-PEG), 1,2-Distearoyl-sn-Glycero-3-Phosphoethanolamine-N-[Methoxy(Polyethylene glycol)-2000] (Ammonium Salt) (DSPE-PEG), 1,2-Dipalmitoyl-sn-Glycero-3-Phosphoethanolamine-N-(Lissamine Rhodamine B Sulfonyl) (Ammonium Salt) (rhodamine-lipid), and 1,2-Dioleoyl-sn-Glycero-3-Phosphoethanolamine-N-(7-nitro-2-1,3-benzoxadiazol-4-yl) (Ammonium Salt) (NBD-lipid). These lipids were purchased from traditional sources.

Using select lipids, the liposomes were prepared by first combining the lipids in chloroform (DPPC and DSPA, or DPPC and DPPA, or DSPC and DSPA, with 5% mole cholesterol, and 2% total PEGylated lipids, DPPE-PEG and DSPE-PEG at 2% mole fractions of the dipalmitoyl- and distearoyl-lipid amounts, respectively) in a 25 ml round bottom flask. The chloroform was evaporated in a rotavapor for 10 minutes at 55° C. and further evaporated under a N₂ stream for 5 minutes. The dried lipid film was then hydrated in 1 ml phosphate buffer (PBS with 1 mM EDTA, pH=7.4) for 2 hours at 50° C. The lipid suspension (10 μmoles total lipid/ml) was then extruded 21 times through two stacked polycarbonate filters of 100 nm pore diameter. Extrusion was carried at 80° C. in a water bath.

In order to evaluate the extent of lipid phase-separation at different pH values, differential scanning calorimetry studies were performed using a VP-DSC Instrument (MicroCal, LLC, Northampton, Mass.). DSC scans of liposome suspensions (0.5 ml, 2.5 mM total lipid) with encapsulated phosphate buffer at the same pH as the surrounding phosphate buffer solution (7.4, 5.5, and 4.0) were performed from 10° C. to 85° C. at a scan rate of 60° C./hr. The thermograph of the corresponding buffer was also acquired at identical conditions, and was subtracted from the excess heat capacity curves. The same scanning conditions were used for the studies with externally added lysozyme (at 3.12 mg protein/ml). Liposome suspensions with encapsulated phosphate buffer (pH 7.4) were incubated for two hours at 37° C. in the presence or absence of lysozyme in solutions of different pH values (7.4, 5.5, and 4.0) before acquisition of thermographs.

### Example 1

The release of encapsulated fluorescent contents, specifically in this example calcein, from PEGylated liposomes, composed of different fractions of DPPC AND DSPA was investigated by calcein quenching efficiency measurements. The lipid film was hydrated in 1 ml phosphate buffer containing 55 mM calcein (pH 7.4, isosmolar to PBS). The unentrapped calcein was removed at room temperature by size exclusion chromatography (SEC) using a Sephadex G-50 column (of 11 cm length) and was eluted with phosphate buffer (1 mM EDTA, pH=7.4). To evaluate the release of calcein from the liposomes, the liposomes containing self-quenching concentrations of calcein (55 mM) were incubated in phosphate buffer or serum supplemented media at different pH values at 37° C. over time. The concentration of lipids for incubation was 0.20 μmoles/ml.

The release of calcein from the liposomes and its dilution in the surrounding solution resulted in an increase in fluorescence due to relief of self-quenching. Calcein release was measured at different time points by adding fixed quantities of liposome suspensions into cuvettes (1 cm path length) containing phosphate buffer (1 mM EDTA, pH 7.4). Calcein fluorescence (ex: 495 nm, em: 515 nm) before and after addition of Triton-X 100, was measured using a Fluoromax-2 spectrofluorometer (Horiba Jobin Yvon, N.J.), and was used to calculate the quenching efficiency defined as the ratio of fluorescence intensities after and before addition of Triton-X 100. The percentage of retained contents with time was calculated as follows:

$$\% calcein content retention = ( \frac{{Q}_{t} - {Q}_{min}}{{Q}_{max} - {Q}_{min}} ) \times 100$$

where, Qₜ is calcein quenching efficiency at the corresponding time point t, Qₘₐₓ is the maximum calcein quenching efficiency in phosphate buffer (at pH 7.4) at room temperature immediately after separation of liposomes by SEC, and Qₘᵢₙ is the minimum quenching efficiency equal to unity.

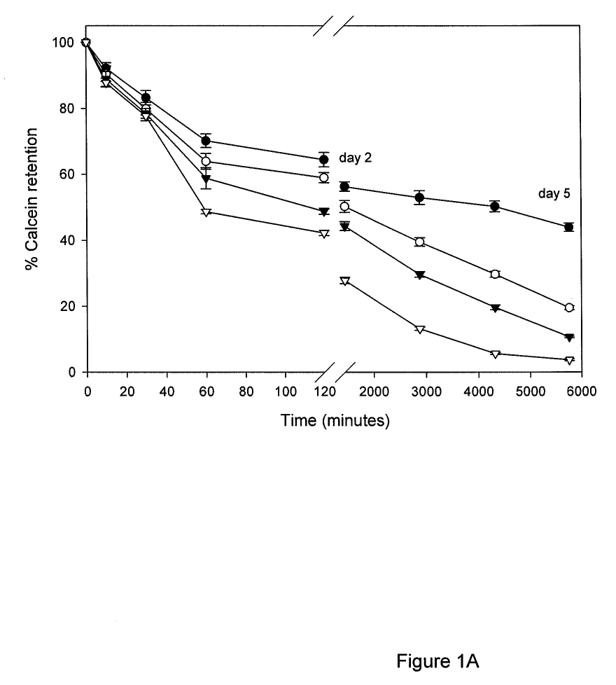

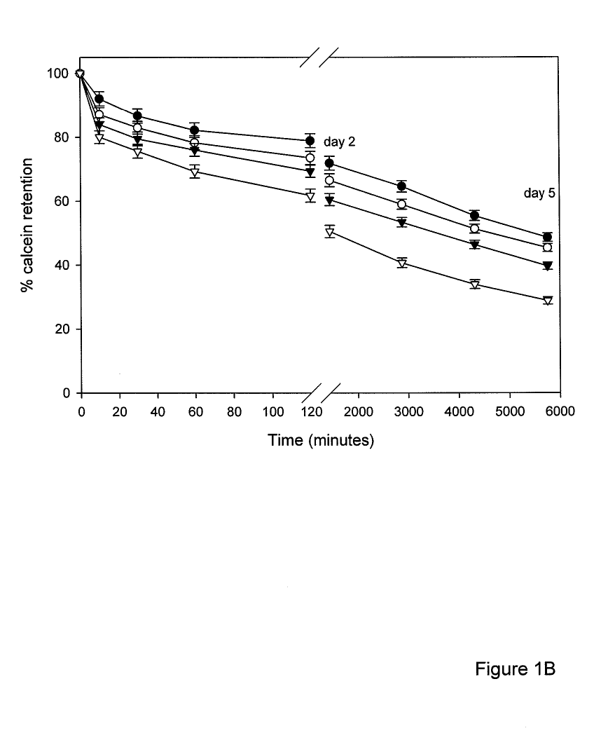

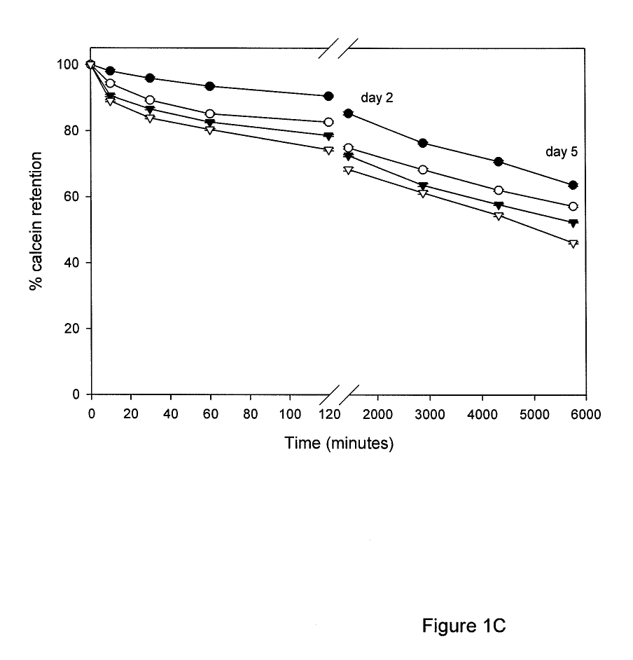

FIGS. 1A, 1B and 1C [pH 7.4 (●), pH 5.5 (∘), pH 5.0 (▾), pH 4.0 (∇)] show that the liposome compositions exhibited pH-dependent behavior as demonstrated by increased content release with decreasing pH in a phosphate buffer solution. As shown in FIG. 1A, the liposomes composed of equimolar DPPC and DSPA lipids exhibited the faster pH-dependent content release. FIGS. 1B and 1C show that a decrease in the fraction of the titratable domain-forming lipid DSPA from 50% mole to 25% mole and 10% mole resulted in higher content retention at every pH value and every time point studied. _After 30 days of incubation, the contents were completely released from all liposome compositions. _PEGylated liposomes composed only of DPPC lipids or only of DSPA lipids exhibited stable retention of 60% or 80%, respectively, of encapsulated contents in phosphate buffer that was not pH-dependent.

Measurable and similar content release (approximately 5 to 10%) was detected within the first 10 minutes of liposome incubation at 37° C. for all liposome compositions and pH values studied. Release of contents during this interval, in addition to phase separation, could be due to differences in osmolarity between the encapsulated solution and the surrounding solvent, or due to the fast heating of liposomes from room temperature to 37° C. that is close to the T_{g} value of the DPPC liposome component (T_{g}=41° C.) resulting in transient membrane destabilization of the DPPC-rich phase due to formation of boundary regions between liquid and solid domains.

Release of encapsulated contents from liposomes containing ‘matching’ hydrocarbon tails (DPPC and DPPA lipids, and DSPC and DSPA lipids) IN PBS is shown on Tables 1A and 1B, respectively.

TABLE 1A

| | 50% mole DPPA | | | | 25% mole DPPA | | | |
|---|---|---|---|---|---|---|---|---|
| Time | pH | | | | pH | | | |
| (minutes) | 7.4 | 5.5 | 5.0 | 4.0 | 7.4 | 5.5 | 5.0 | 4.0 |
| 0 | 100 ± 0 | 100 ± 0 | 100 ± 0 | 100 ± 0 | 100 ± 0 | 100 ± 0 | 100 ± 0 | 100 ± 0 |
| 10 | 69 ± 3 | 66 ± 1 | 64 ± 2 | 59 ± 2 | 86 ± 4 | 80 ± 3 | 86 ± 3 | 80 ± 4 |
| 30 | 64 ± 2 | 63 ± 2 | 58 ± 2 | 59 ± 2 | 86 ± 4 | 77 ± 2 | 85 ± 4 | 80 ± 4 |
| 60 | 61 ± 2 | 61 ± 4 | 56 ± 1 | 60 ± 2 | 79 ± 5 | 76 ± 1 | 79 ± 3 | 79 ± 6 |
| 120 | 61 ± 8 | 62 ± 2 | 57 ± 4 | 55 ± 2 | 78 ± 4 | 76 ± 4 | 76 ± 4 | 77 ± 4 |
| 1440 (day 2) | 47 ± 3 | 41 ± 1 | 41 ± 1 | 35 ± 1 | 64 ± 1 | 59 ± 1 | 61 ± 2 | 58 ± 1 |
| 5760 (day 5) | 28 ± 1 | 20 ± 1 | 22 ± 1 | 14 ± 1 | 36 ± 2 | 28 ± 2 | 29 ± 4 | 27 ± 6 |

TABLE 1B

| | 50% mole DSPA | | | | 25% mole DSPA | | | |
|---|---|---|---|---|---|---|---|---|
| Time | pH | | | | pH | | | |
| (minutes) | 7.4 | 5.5 | 5.0 | 4.0 | 7.4 | 5.5 | 5.0 | 4.0 |
| 0 | 100 ± 0 | 100 ± 0 | 100 ± 0 | 100 ± 0 | 100 ± 0 | 100 ± 0 | 100 ± 0 | 100 ± 0 |
| 10 | 69 ± 2 | 63 ± 1 | 64 ± 3 | 56 ± 2 | 89 ± 4 | 87 ± 6 | 86 ± 3 | 76 ± 3 |
| 30 | 62 ± 1 | 64 ± 1 | 57 ± 2 | 49 ± 1 | 83 ± 3 | 83 ± 3 | 78 ± 3 | 74 ± 3 |
| 60 | 62 ± 4 | 58 ± 1 | 61 ± 1 | 51 ± 1 | 75 ± 3 | 68 ± 6 | 66 ± 2 | 71 ± 5 |
| 120 | 63 ± 1 | 58 ± 5 | 49 ± 3 | 50 ± 4 | 72 ± 2 | 63 ± 3 | 69 ± 3 | 66 ± 5 |
| 1440 (day 2) | 53 ± 1 | 46 ± 4 | 37 ± 0 | 32 ± 1 | 67 ± 3 | 66 ± 2 | 63 ± 2 | 62 ± 2 |
| 5760 (day 5) | 39 ± 1 | 17 ± 1 | 13 ± 1 | 13 ± 0 | 60 ± 2 | 59 ± 1 | 58 ± 3 | 52 ± 2 |

As can be seen, both types of liposomes exhibited an initial drop in content retention within the first 10 minutes of incubation that was dependent on the fraction of ‘titratable lipid’ (DPPA or DSPA, Table 1A and Table 1B, respectively) and not on pH. Decrease of the fraction of DPPA or DSPA lipid resulted in higher overall content retention. For the first 24 hours of incubation, all liposome compositions exhibited content release profiles that were not pH-dependent.

TABLE 2

| | Percentage of calcein retention | | | |
|---|---|---|---|---|
| Time | pH | | | |
| (minutes) | 7.4 | 5.5 | 5.0 | 4.0 |
| 0 | 100 ± 0 | 100 ± 0 | 100 ± 0 | 100 ± 0 |
| 10 | 92 ± 2 | 85 ± 3 | 86 ± 2 | 82 ± 2 |
| 30 | 86 ± 3 | 80 ± 3 | 79 ± 4 | 73 ± 2 |
| 60 | 81 ± 2 | 74 ± 2 | 71 ± 4 | 66 ± 2 |
| 120 | 78 ± 3 | 71 ± 2 | 69 ± 3 | 62 ± 3 |
| 1440 (day 2) | 58 ± 2 | 53 ± 3 | 49 ± 3 | 37 ± 2 |
| 5760 (day 5) | 30 ± 4 | 25 ± 3 | 23 ± 6 | 13 ± 9 |

### Example 2

For dextran retention measurements, the dried lipid film was hydrated with 1 ml phosphate buffer containing 3 kDa dextrans (0.5 mg/ml) or 10 kDa dextrans (0.62 mg/ml). To evaluate the release of dextrans from liposomes, liposomes containing fluorescent dextrans of variable molecular weight (3 kDa and 10 kDa) were incubated in phosphate buffer at different pH values at 37° C. over time. The concentration of lipids during incubation was 1.25 μmoles/ml in phosphate buffer in order to increase the concentration of encapsulated dextrans and to improve the efficacy of their detection due to low dextran entrapment efficiency by liposomes. At various time points, liposome fractions were removed from the parent liposome suspension and released dextrans were separated from liposomes by SEC using a Sepharose 4B column (of 11 cm length) eluted with phosphate buffer (1 mM EDTA, pH=7.4) at room temperature. Free dextrans and dextrans encapsulated in liposomes were quantitated by fluorescence spectroscopy (ex: 595 nm, em: 615 nm).

Smaller dextrans (MW=3 KDa, approximately 4.4 nm in diameter) were released in a pH-dependent manner only from liposomes that contained the maximum fraction (50% mole) of DSPA lipid. Large dextran particles (MW=10 KDa, approximately 5.7 nm in diameter) were not released from any composition at all conditions studied indicating an upper cutoff size of the domain/non-domain interface.

To evaluate the extent of liposomal membrane ‘discontinuities’ caused by lipid-phase separation with decreasing pH-values, fluorescently labeled dextrans of molecular sizes larger than calcein, were encapsulated in liposomes and their pH-dependent release from liposomes was measured.

Table 3 shows that 3 kDa dextans are released from equimolar DPPC and DSPA liposomes in a pH-dependent manner with comparable kinetics to those of calcein. Decreasing the fraction of the titratable domain-forming lipid DSPA (25% and 10% mole DSPA content, Table 3) increased retention of contents and exhibited almost loss of pH-dependent content release. Larger dextrans (10 kDa) were stably retained (>93%) by all liposome compositions for 30 days (data not shown).

TABLE 3

| | 50% mole DSPA | | | | 25% mole DSPA | |
|---|---|---|---|---|---|---|
| Time | pH | | | | pH | |
| (minutes) | 7.4 | 5.5 | 5.0 | 4.0 | 7.4 | 5.5 |
| 0 | 100 ± 0 | 100 ± 0 | 100 ± 0 | 100 ± 0 | 100 ± 0 | 100 ± 0 |
| 10 | 95 ± 2 | 85 ± 3 | 86 ± 3 | 79 ± 4 | 97 ± 4 | 99 ± 0 |
| 30 | 93 ± 0 | 81 ± 4 | 80 ± 2 | 71 ± 4 | 98 ± 1 | 94 ± 10 |
| 60 | 89 ± 1 | 79 ± 3 | 75 ± 4 | 68 ± 2 | 96 ± 2 | 98 ± 1 |
| 1440 (day 2) | 88 ± 2 | 68 ± 3 | 66 ± 2 | 61 ± 2 | 97 ± 2 | 94 ± 6 |
| 5760 (day 5) | 68 ± 2 | 59 ± 3 | 60 ± 3 | 51 ± 3 | 95 ± 2 | 94 ± 2 |
| | 25% mole DSPA | | 10% mole DSPA | | | |
| Time | pH | | pH | | | |
| (minutes) | 5.0 | 4.0 | 7.4 | 5.5 | 5.0 | 4.0 |
| 0 | 100 ± 0 | 100 ± 0 | 100 ± 0 | 100 ± 0 | 100 ± 0 | 100 ± 0 |
| 10 | 98 ± 1 | 94 ± 1 | 95 ± 3 | 97 ± 1 | 98 ± 1 | 96 ± 2 |
| 30 | 93 ± 8 | 93 ± 5 | 90 ± 3 | 93 ± 2 | 95 ± 2 | 94 ± 6 |
| 60 | 97 ± 0 | 95 ± 1 | 91 ± 2 | 94 ± 4 | 97 ± 0 | 95 ± 1 |
| 1440 (day 2) | 95 ± 2 | 93 ± 6 | 93 ± 2 | 95 ± 3 | 96 ± 4 | 96 ± 2 |
| 5760 (day 5) | 93 ± 1 | 90 ± 3 | 91 ± 4 | 94 ± 4 | 96 ± 1 | 94 ± 0 |

### Example 3

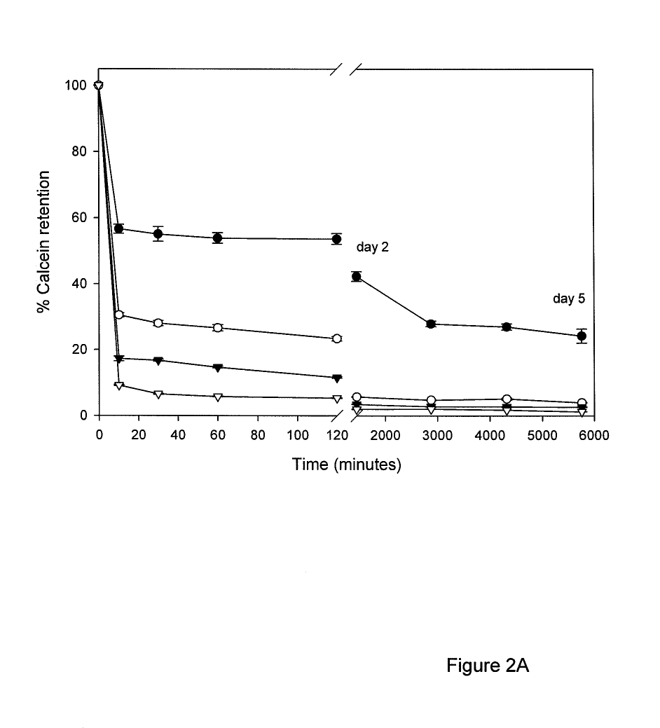

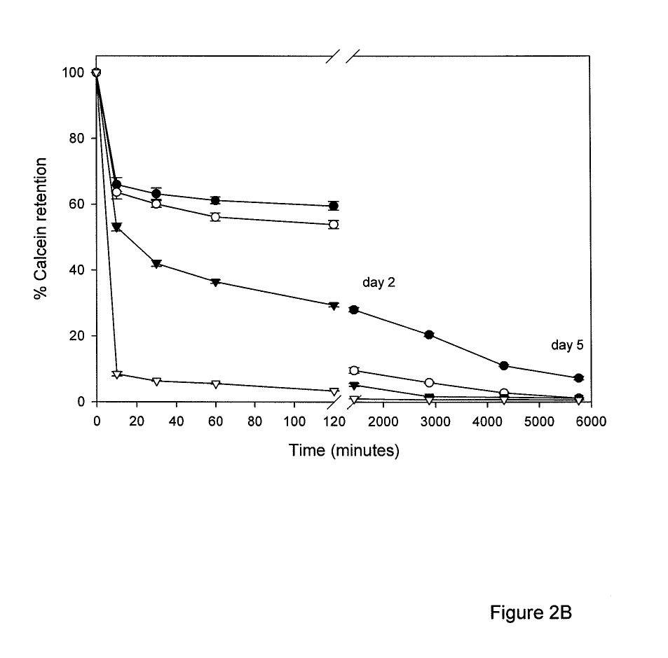

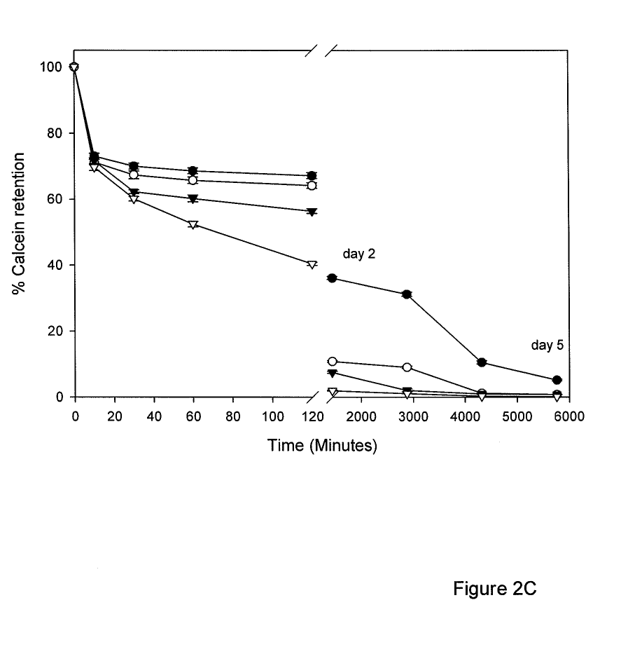

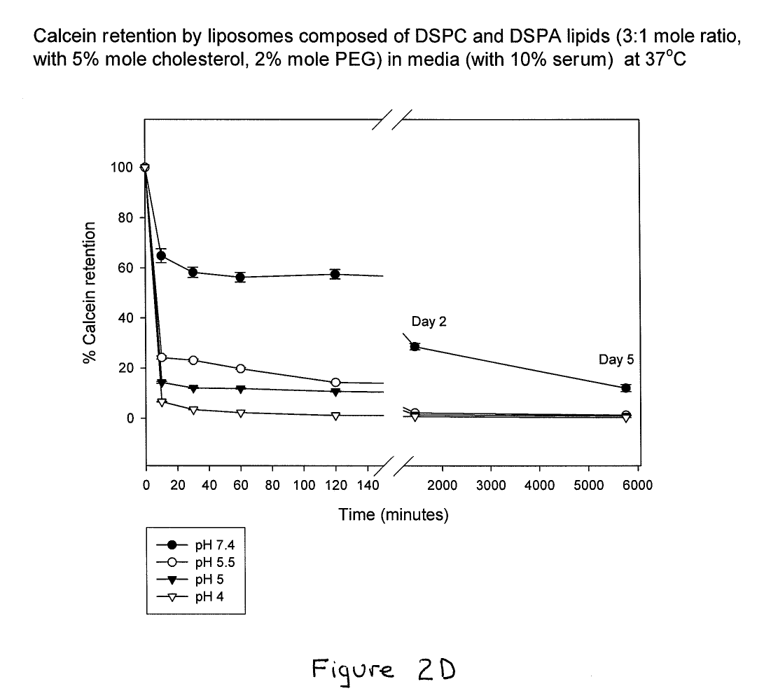

FIGS. 2A, 2B and 2C [pH 7.4 (●), pH 5.5 (∘), pH 5.0 (▾), pH 4.0 (∇)] show the pH-dependent release of fluorescent contents (calcein) from liposomes in 10% serum supplemented media. Liposomes containing larger fractions of the titratable domain-forming lipid DSPA exhibited greater and faster release of contents, at every pH value studied. Release kinetics was significantly faster compared to measurements in phosphate buffer. In particular, liposomes containing equimolar DPPC and DSPA lipids released within 30 minutes of incubation at the ‘endosomally relevant’ pH values of 5.5 and 5.0, 49% and 70% of their contents, respectively, compared to pH 7.4. The observed content release within the first 10 minutes of incubation appears to be pH-dependent in addition to a possible thermal or osmotic effect on membrane destabilization at the onset of incubation as mentioned above.

### Example 4

FIG. 2D shows that release of fluorescent contents (calcein) from liposomes (25% DSPA lipid and 75% DSPC lipid) in the presence of 10% serum proteins in media exhibit pH sensitive release of contents. In less than 20 minutes in acidic pH (5.5 and 5.0, corresponding to early and late endosomal environments), these liposomes release a very significant fraction of the encapsulated contents. At the same time, these liposomes stably contain their contents at pH 7.4, which corresponds to the pH during blood circulation where contents are required to be retained by the liposomes.

### Example 5

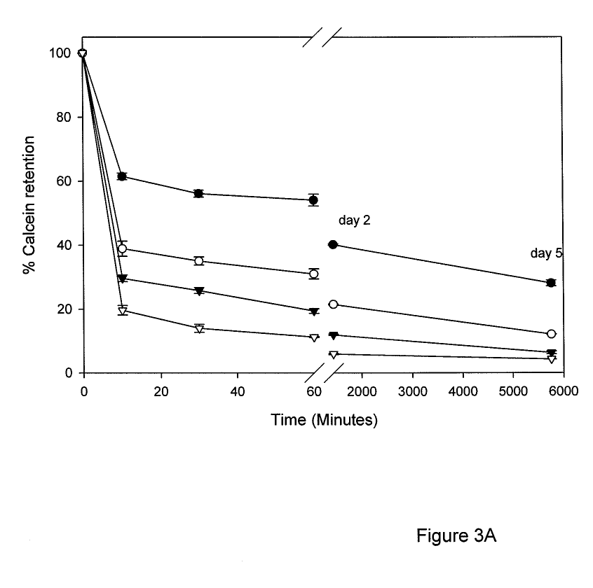

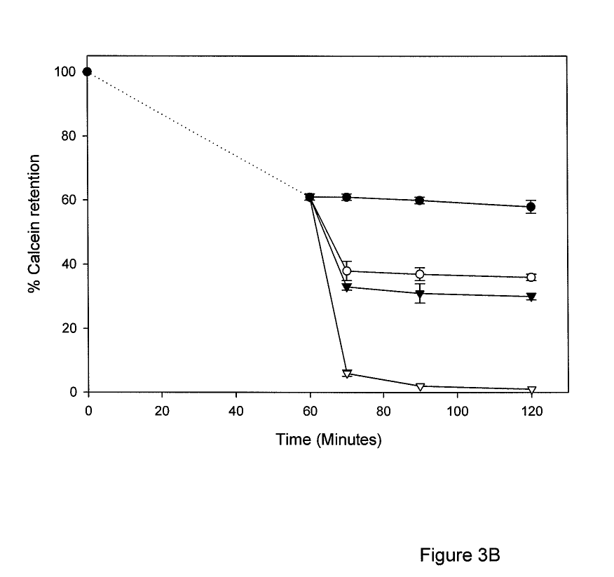

FIGS. 3A and 3B show the percentage of calcein retention as a function of pH [pH 7.4 (●), pH 5.5 (∘), pH 5.0 (▾), pH 4.0 (∇)] by liposomes composed of 75% mole DPPC and 25% mole DSPA (with 5% mole cholesterol and 2% mole PEGylated lipids), incubated in 60% serum supplemented media at 37° C. FIG. 3A shows the content release over 5 days and FIG. 3B shows content release upon pH decrease (indicated by black arrow) after a 60 minute preincubation period at pH 7.4. The error bars correspond to standard deviations of repeated measurements 2 liposome preparations, 2 samples per preparation per time point.

FIG. 3A shows that release of fluorescent contents (calcein) from liposomes (25% mole DSPA lipid and 75% DPPC) in the presence of physiologic serum concentrations (60% serum at 370 Celsius) exhibit similar dependence on pH as in low serum containing media. For short incubation times (less than one hour), liposomes exhibited increased content release at lower pH values. pH-sensitivity was observed in all three lipid ratios studied (data not shown for 50% and 10% mole DSPA). The extent of released contents and the initial release rates in serum supplemented media increase with decreasing pH and with increasing serum concentration.

### Example 6

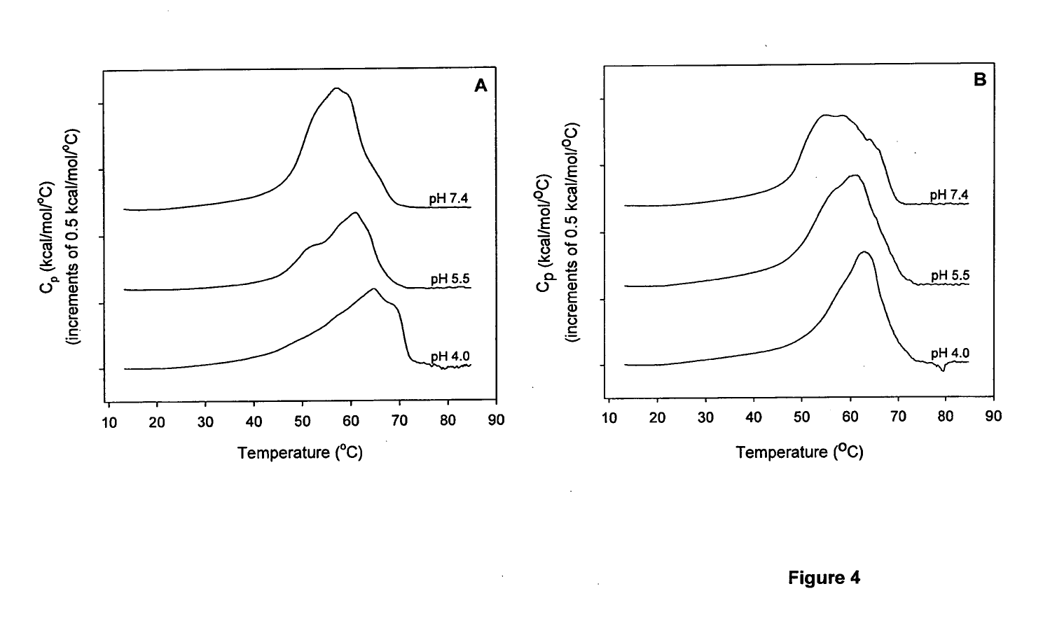

FIG. 4A shows the thermal scans of PEGylated liposomes composed of equimolar DPPC and DSPA lipids (with 5% mole cholesterol) in phosphate buffer after two hours of incubation at 37° C. Enhancement on the contributions from thermal transitions at higher temperatures is observed with decreasing pH values from 7.4 to 4.0 (pH values studied: 7.4, 5.5, and 4.0). Higher thermal transitions containing multiple peaks at lower pH values suggest increasing formation of lipid phases that should be rich in clustered (protonated) DSPA lipids. FIG. 4B shows that longer incubation (four days) of liposome suspensions at 37° C. resulted in similar shifts toward higher thermal transitions with decreasing pH, but with increased calorimetric peaks compared to the earlier time point (FIG. 4A). Decrease of the fraction of the titratable domain-forming lipid DSPA (25% and 10% mole) resulted in similar contributions from thermal transitions but at lower temperatures for all pH values studied (7.4, 5.5, and 4.0).

Lipid phase-separation in the presence of serum should be caused by clustering of charged DSPA lipids that form domains under the electrostatic attraction of charged serum proteins that are adhering onto the liposome membranes. In serum supplemented media, a similar mechanism of domain formation should account for the observed pH-sensitivity of the liposomes studied. Although liposome aggregation was detected with decreasing solution pH and with time, no fusion was detected among the aggregated liposomes. In 60% serum supplemented media, liposomes containing 25% mole DSPA lipids exhibited pH-dependent release of contents and they significantly retained their encapsulated contents at pH 7.4 even after 24 hours of incubation. The pH-sensitive release kinetics is fast and comparable to the kinetics of endocytosis. This is supported by the DSC thermographs using lysozyme as a model protein.

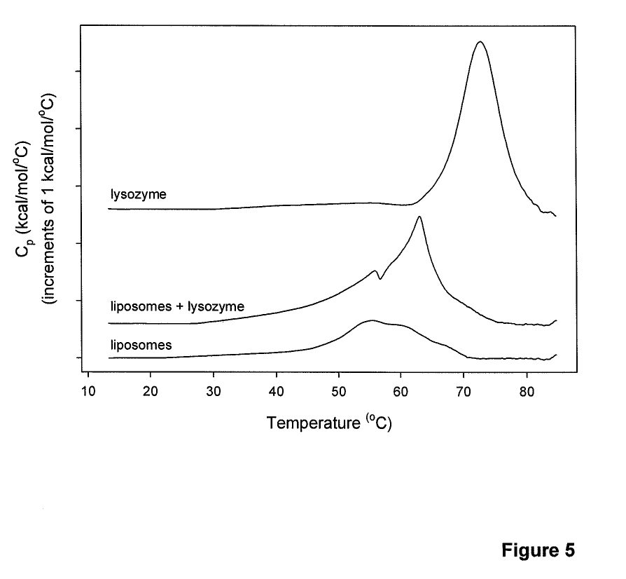

In the presence of serum proteins the pH-dependent content release from liposomes should be due to the phase separation of charged DSPA lipids caused by adsorption of charged proteins onto the liposome surface. To test this hypothesis, lysozyme was added to liposome suspensions and the thermal transitions were evaluated. Lysozyme was used as a model protein because of its high denaturation temperature (72° C.) that should not significantly interfere with the temperature window defined by the thermal transitions of DPPC and DSPA lipids (41° C. and 75° C., respectively). FIG. 5 shows the thermographs of equimolar DPPC- and DSPA-containing liposomes at neutral pH in the presence and absence of lysozyme. The split of the main transition peak of liposomes in the presence of lysozyme probably suggests lateral phase separation caused by the deprotonated (negatively charged) DSPA lipids upon protein adsorption. The shift of the protein denaturation peak to lower temperatures also suggests adsorption of lysozyme onto the lipid membrane. Thermographs at lower pH values (5.5 and 4.0) in the presence of lysozyme resulted in multiple splits on the broad lipid transition peak that could probably be correlated to the more than one transition temperatures that were detected on free lysozyme at the corresponding pH values (data not shown).

### Example 7

The measured average liposome size of equimolar DPPC- and DSPA-containing liposomes was 262±57 nm in diameter at pH 7.4 immediately after liposome extrusion in phosphate buffer. Table 4 shows that incubation of liposomes at 37° C. in phosphate buffer at pH values ranging from 7.4 to 4.0, does not cause changes on the liposome size distributions over time (three days), suggesting that no liposome aggregation occurs. As shown in Table 4, in 10% serum supplemented media, liposome aggregation was observed at the lower pH values and increased with time and with increasing DSPA content (Table 4).

TABLE 4

| | 50% mole DSPA | | | 25% mole DSPA | | | 10% mole DSPA | | |
|---|---|---|---|---|---|---|---|---|---|
| Time | pH | | | pH | | | pH | | |
| (days) | 7.4 | 5.5 | 4.0 | 7.4 | 5.5 | 4.0 | 7.4 | 5.5 | 4.0 |

phosphate buffer

| | | | | | | | | | |
|---|---|---|---|---|---|---|---|---|---|
| 1 | 269 ± 57 | 249 ± 61 | 236 ± 34 | 236 ± 37 | 261 ± 31 | 245 ± 37 | 213 ± 37 | 211 ± 41 | 234 ± 38 |
| 2 | 235 ± 45 | 215 ± 36 | 246 ± 48 | 246 ± 29 | 255 ± 36 | 268 ± 39 | 212 ± 33 | 216 ± 35 | 204 ± 27 |
| 3 | 242 ± 38 | 247 ± 31 | 259 ± 48 | 249 ± 30 | 229 ± 21 | 251 ± 35 | 240 ± 25 | 241 ± 28 | 234 ± 23 |

10% serum

| | | | | | | | | | |
|---|---|---|---|---|---|---|---|---|---|
| 1 | 209 ± 41 | 200 ± 73 | 369 ± 82 | 136 ± 20 | 158 ± 29 | 275 ± 38 | 148 ± 21 | 159 ± 27 | 252 ± 36 |
| 2 | 273 ± 60 | 245 ± 50 | 466 ± 59 | 141 ± 23 | 184 ± 28 | 383 ± 20 | 152 ± 16 | 180 ± 24 | 354 ± 35 |
| 3 | 272 ± 52 | 311 ± 67 | 453 ± 91 | 154 ± 25 | 221 ± 32 | 428 ± 31 | 148 ± 29 | 207 ± 29 | 354 ± 27 |

### Example 8

Interliposome fluorescence resonance energy transfer (FRET) (ex: 496 nm, em: 590 nm) was used to evaluate possible fusion among liposomes at decreasing pH values-. Two populations of liposomes were prepared. One population contained NBD-lipids (energy donor) and rhodamine-lipids (energy acceptor) each at 0.5% mole fraction. The other liposome population did not contain fluorophores. Liposome fractions from each population were mixed at equal volumes at various pH values and their fluorescence intensity was compared to samples containing only liposomes from the fluorescent liposome population. Liposome fusion increases the effective distances between fluorophores, resulting in lower emission intensities of the energy acceptor. The fluorescent intensities of samples were monitored over time and the ratios of the intensities of samples containing both liposome populations were normalized by the intensities of samples containing only the fluorescent liposomes to account for quenching or photo-bleaching effects unrelated to fusion.

For the equimolar DPPC- and DSPA-containing liposomes, no change in the FRET intensities was observed over time (four days), both in phosphate buffer and in 10% serum supplemented media for all pH values studied (7.4, 5.5, 4.0) suggesting that no fusion occurs even after liposome aggregation in media.

### Example 9

Zeta potential measurements verified decrease of the surface negative charge of liposome membranes with decreasing pH. To better characterize the discontinuities along the domain/non-domain interface, the pH-dependent release of fluorescent dextrans of larger sizes was also studied. The zeta potential of dextrans was measured at the pH values studied and was found to range between −14.1 and −6.5 mV, and −3.6 and −4.1 mV for 3 KDa and 10 kDa dextrans, respectively, suggesting that a release mechanism other than direct diffusion across the membrane transient discontinuities would be highly unlikely.

The zeta potential of liposomes containing different fractions of the titratable domain-forming lipid DSPA was measured in phosphate buffer at different pH values (7.4, 5.5, and 4.0) using a Zetasizer ZS90 (Malvern instruments, Worcestershire, UK). Measurements were performed in 10 mM PBS with no salt. For these measurements, liposomes (1 mM total lipid) were prepared without grafted PEG-chains on their surface to allow for closer approach of the plane of shear to the physical surface of lipid membranes. Exclusion of PEGylated phosphatidyl ethanolamine (pKa=1.7) at 2% mole fraction is not expected to significantly influence the values of zeta potential (except, probably, for the lower concentration (10% mole) of DSPA).

Table 5 shows the measured zeta potential values of liposomes containing different fractions of the titratable domain-forming lipid DSPA at pH 7.4, 5.5 and 4.0. Liposomes exhibited decreasing values of negative zeta potential with decreasing pH and with decreasing DSPA content.

TABLE 5

| | 50% mole DSPA | 25% mole DSPA | 10% mole DSPA |
|---|---|---|---|
| pH | Zeta potential (mV) | Zeta potential (mV) | Zeta potential (mV) |
| 7.4 | −68.5 ± 0.5 | −50.4 ± 0.6 | −41.5 ± 0.4 |
| 5.5 | −65.2 ± 0.7 | −41.7 ± 0.5 | −37.1 ± 0.5 |
| 4.0 | −55.3 ± 0.5 | −38.6 ± 0.7 | −32.1 ± 0.2 |

### Example 10

Dynamic light scattering (DLS) of liposome suspensions was studied with an N4 plus autocorrelator (Beckman-Coulter, Fullerton, Calif.), equipped with a 632.8 nm He—Ne laser light source. Scattering was detected at 23.0, 30.2, 62.6 and 90°. Particle size distributions at each angle were calculated from autocorrelation data analysis by CONTIN. The average liposome size was calculated to be the y-intercept at zero angle of the measured average particle size values versus sin²(θ). All buffer solutions used were filtered with 0.22 μm filters just before liposome preparation. The collection times for the autocorrelation data were 1 to 10 minutes. Liposomes were incubated in different solutions (phosphate buffer and serum supplemented media) at 37° C., and liposome fractions were removed from the parent liposome suspension over time, and were diluted in phosphate buffer (7.4) before measurement.

Liposome membranes composed of two types of lipids (DPPC and DSPA with 5% mole cholesterol) exhibit lipid phase-separation as a response to decreasing pH values and resulting in content release. However, despite the presence of these relatively large discontinuities, these liposomes retain their structure in solution and do not collapse into larger aggregates as shown by DLS measurements. These studies qualitatively correlate the extent of non-ionized domain-forming DSPA lipids to the extent of lipid phase separation (measured by DSC). Increased incubation times resulted in higher calorimetric peaks (higher overall enthalpy changes) that potentially indicates increased number of lipids associated with the phase-separated domains or the formation of clustered phase-separated domains. Clustering of domains decreases the total domain/non-domain interface and should result in decrease of the number of thermal contributions of lower thermal transition temperatures that originate from lipids residing in the less ordered domain/non-domain interface.

The foregoing detailed description of the embodiments and the appended figures have been presented only for illustrative and descriptive purposes. They are not intended to be exhaustive and are not intended to limit the scope and spirit of the invention. The embodiments were selected and described to best explain the principles of the invention and its practical applications. One skilled in the art will recognize that many variations can be made to the invention disclosed in this specification without departing from the scope and spirit of the invention.

What is claimed is:

1. A liposome composition for delivering a biologically active agent, comprising:
a) at a least first rigid lipid and a second rigid lipid each having a head group and a hydrophobic tail;
b) a polyethyleneglycol-linked lipid having tail matching at least a portion of the first or the second lipid; and
c) an entrapped biologically active agent,
wherein the first lipid is a zwitterionic lipid and the second lipid has a titratable head group, and the composition is adapted to release the entrapped biologically active agent at a certain pH.

2. The composition of claim 1, further comprising a stabilizing component.

3. The composition of claim 1, wherein the first and second lipids are present in equal proportions.

4. The composition of claim 1, wherein the first and second lipids are present in unequal proportions.

5. The composition of claim 1, wherein the first and second lipids each have Tg>37° Celsius.

6. The composition of claim 1, wherein the certain pH is lower than about 7.

7. The composition of claim 1, further comprising a coating on membrane surfaces of the liposomes, wherein the coating preferentially associates with a specific target cell.

8. The composition of claim 1, wherein the biologically active agent is toxic to cancer cells.

9. The composition of claim 1, wherein the head group of at least one of the lipids is negative at a neutral pH.

10. The composition of claim 1, wherein the first lipid is DSPC or DPPC and the second lipid is DSPA.

11. A method for increasing accumulation of a biologically active agent proximal to a cell having a acidic environment, comprising
a) administering liposomes comprising at a least first lipid and a second lipid each having a head group and a hydrophobic tail, a polyethyleneglycol-linked lipid having a tail matching at least a portion of the first or the second lipid, and an entrapped biologically active agent; and
b) allowing the liposomes to release the entrapped biologically active agent in the acidic environment,
wherein the first lipid is a zwitterionic lipid and the second lipid has a titratable head group, and the liposome are adapted to release the entrapped biologicially active agent at a certain pH, and
whereby the release of the biologically active agent is effective to achieve an increase in the accumulation of the biologically active agent in the acidic environment.

12. The method as claimed in claim 11, wherein the first and second lipid each have Tg>37° C.

13. The method as claimed in claim 11, wherein the liposomes are capable of releasing the entrapped biologically active agent in an environment is less than about 7.

14. The method as claimed in claim 11, wherein the liposomes are capable of releasing the entrapped biologically active agent at metastatic tumors with developed vasculature

15. The method as claimed in claim 11, wherein the first lipid is DSPC or DPPC and the second lipid is DSPA.

16. A method for administering a biologically active agent comprising:
a) selecting a liposome comprising at a least first rigid lipid and a second rigid lipid each having a head group and a hydrophobic tail and a polyethyleneglycol-linked lipid having a side chain matching at least a portion of the first or the second lipid, wherein the first lipid is a zwitterionic lipid and the second lipid has a titratable head group; and
b) preparing a liposome composition with at least the first rigid lipid, the second rigid lipid, and the polyethyleneglycol-linked lipid;
c) preparing a therapeutic liposome by combining the liposome composition with a biologically active agent so that the biologically active agent is entrapped within the liposome composition, and
whereby the therapeutic liposome is adapted to release the entrapped biologically active agent at a certain pH; and
d) administering the therapeutic liposome to a subject.

17. The method as claimed in claim 16, wherein the liposome composition is prepared to release the biologically active agent in an environment with a pH of less than 7:

18. The method as claimed in claim 16, wherein the first and second lipids are selected so that the liposome composition will release the entrapped biologically active agent at a desired pH.

19. The method as claimed in claim 16, wherein the head group on the first lipid is selected so that the liposome composition will release the entrapped biologically active agent at a desired pH.

20. The method as claimed in claim 16, wherein the head group on the second lipid is selected so that the liposome composition will release the entrapped biologically active agent at a desired pH.
